(19) United States
(12) Patent Application Publication
(10) Pub. No.: US 2026/0244992 A1
(43) Pub. Date: Aug. 20, 2026

(54) MULTIMODAL EMOTION RECOGNITION BRAIN-COMPUTER INTERFACE INSTRUMENT BASED ON CONTRASTIVE LEARNING GATED NETWORK

(71) Applicant: TIANJIN UNIVERSITY, Tianjin (CN)

(72) Inventors: Biao SUN, Tianjin (CN); Xiaochen REN, Tianjin (CN); Jing YANG, Tianjin (CN); Chi YAO, Tianjin (CN); Chen LIU, Tianjin (CN); Minpeng XU, Tianjin (CN)

(73) Assignee: TIANJIN UNIVERSITY, Tianjin (CN)

(21) Appl. No.: 19/538,822

(22) Filed: Feb. 12, 2026

Related U.S. Application Data
(63) Continuation of application No. PCT/CN2025/088228, filed on Apr. 10, 2025.

(30) Foreign Application Priority Data
Feb. 20, 2025 (CN) 202510190843.0

(51) Int. Cl. G06N 20/00

(52) U.S. Cl. CPC G06N 20/00

(57) ABSTRACT

A multimodal emotion recognition instrument includes an electroencephalogram (EEG) data collection apparatus configured to collect EEG data, an eye movement data collection apparatus configured to collect eye movement data, and a multimodal data processing apparatus. Firstly, a hardware collection apparatus is designed through EEG and eye signals, so as to ensure efficient and accurate acquisition of data; and secondly, a multimodal contrastive learning gated network is designed to improve the accuracy of emotion recognition.

### CROSS-REFERENCE TO RELATED APPLICATIONS

This application is a continuation of International Application No. PCT/CN2025/088228, filed on Apr. 10, 2025, which claims priority to Chinese Patent Application No. 202510190843.0, filed on Feb. 20, 2025. All of the aforementioned applications are incorporated herein by reference in their entireties.

### TECHNICAL FIELD

The present disclosure belongs to the field of brain-computer interfaces, and in particular, relates to a multimodal emotion recognition brain-computer interface instrument based on a contrastive learning gated network.

### BACKGROUND

With the development of computer and information technologies, machines with an emotion recognition ability can significantly improve the user experience of human-computer interaction and provide smoother and more natural interfaces for human-computer interaction. In this context, emotion recognition has received widespread attention from academia and industry, and has been widely applied in intelligent driving, medical care, intelligent robots, and other fields. The input modes used in the existing emotion recognition research are generally divided into physiological signals and non-physiological signals, wherein the physiological signals are not influenced by human subjective consciousness and can more truly and reliably reflect human emotion changes.

The research on emotion recognition using EEG and peripheral physiological signals has a long history. However, traditional deep learning feature extractors usually only focus on specific feature scales, such as single spatial domain, temporal domain or frequency domain features. This limitation makes the extracted feature range not comprehensive enough, and possibly overlooks the key multi-dimensional information in emotion recognition. Therefore, comprehensive multi-scale features are crucial for improving the accuracy of emotion recognition. Effectively integrating these multi-scale features can help capture more detailed emotion changes, thereby improving the performance and robustness of models.

Traditional emotion recognition methods mainly rely on unimodal data, but human emotion expression is diverse, and different emotion information is contained in different forms of physiological signals, so it is necessary to combine multiple physiological signals to express emotions. More comprehensive emotion information is acquired according to the difference and complementarity of different modes.

In existing multimodal emotion recognition, a common method is to simply splice or fuse features from different modes. This simple feature fusion mode cannot fully capture the complex relationships among various modes, thereby affecting the performance of models. In order to effectively fuse multi-source data features, some researches use contrastive learning methods to learn the similarity and difference among different modes, so as to extract and align semantic information in different modal data.

In addition, in multimodal tasks, each mode has unique advantages and information. How to flexibly select the optimal mode according to specific situations and effectively switch between a dominant mode and multiple modes is an important challenge. The existing multimodal emotion recognition methods are not flexible enough in modal selection and switching, resulting in insufficient utilization of all valuable modal information in some samples.

It can be seen that in multimodal emotion recognition, more attention needs to be paid to the diversity and complementarity of features, and more efficient dynamic decision mechanisms need to be explored to achieve a more accurate and natural human-computer interaction experience.

### SUMMARY

In order to overcome the problem of inaccurate multimodal emotion recognition in the prior art, the present disclosure provides a multimodal emotion recognition instrument based on a contrastive learning gated network, which comprehensively improves the performance of emotion recognition through dual optimization of hardware and algorithms. The present disclosure firstly combines electroencephalogram (EEG) and eye signals to design a hardware collection apparatus, so as to ensure efficient and accurate acquisition of data. Secondly, a multimodal contrastive learning gated network (MCGNet) is designed to improve the accuracy of emotion recognition.

The present disclosure relates to a multimodal emotion recognition brain-computer interface instrument based on a contrastive learning gated network. The instrument is composed of an EEG data collection apparatus, an eye movement data collection apparatus, and a multimodal data processing apparatus, wherein the multimodal data processing apparatus includes a microprogrammed control unit (MCU), a neural network accelerator, a data bus, a data memory, and an output interface. The EEG data collection apparatus is configured to collect EEG data, the eye movement data collection apparatus is configured to collect eye movement data, and the collected EEG data and eye movement data are stored into the data memory through the control of the MCU. Further, the MCU controls the neural network accelerator to acquire the EEG data and eye movement data from the data memory to train the multimodal contrastive learning gated network.

Further, the EEG data collection apparatus is composed of a power source, a main control chip, and an analog-to-digital converter.

Further, the eye movement data collection apparatus is composed of binocular infrared cameras, and the infrared cameras can capture clear eye images in low-light or no-light environments to avoid visible light interference.

In order to verify the practical effect of a multimodal collection helmet in emotion recognition, a multimodal emotion recognition method MCGNet combining EEG signals and eye movement signals is performed in the multimodal emotion recognition brain-computer interface instrument provided in the present disclosure. Specifically, the MCU controls the neural network accelerator to acquire the EEG data and eye movement data from the data memory to train the multimodal contrastive learning gated network. The training includes the following three modules: a unimodal feature encoder pre-training module, a multimodal contrastive learning training module, and a gated structure training module.

The unimodal feature encoder pre-training module is configured to respectively input EEG primary features and eye movement primary features into an EEG feature encoder and an eye movement feature encoder, and respectively input encoding results into a classifier to respectively predict unimodal emotion categories.

The multimodal contrastive learning training module is configured to respectively input EEG primary features and eye movement primary features into feature encoders of the unimodal feature encoder pre-training module to obtain multimodal features EEG and Eye, then respectively use an EEG similarity mapper and an EEG dissimilarity mapper as well as an eye similarity mapper and an eye dissimilarity mapper to decompose EEG and Eye signals, fuse decomposition results, and perform intra-sample contrastive learning and inter-sample contrastive learning.

The gated structure training module is configured to fuse EEG primary features and eye movement primary features and then input the fused EEG primary features and eye movement primary features into a gated network, the gated network generating a two-dimensional vector as an output to select and activate different expert network branches to complete emotion classification tasks.

The present disclosure utilizes contrastive representation learning and contrastive feature decomposition to enhance the representation of multimodal information. In addition, in order to solve the problem that sometimes the fusion of multimodal information can introduce noise interference, resulting in lower accuracy than a unimodal manner, the gated structure is introduced, and a unimodal or multimodal manner and corresponding expert networks thereof are selected according to sample features to improve the accuracy of emotion recognition.

### BRIEF DESCRIPTION OF THE DRAWINGS

In order to describe the technical solutions in the embodiments of the present disclosure more clearly, the following briefly introduces the accompanying drawings required for describing the embodiments or prior art. Obviously, the accompanying drawings described below are only some embodiments of the present disclosure. Those skilled in the art can also obtain other accompanying drawings according to these accompanying drawings without any creative work.

FIG. 1 is a system diagram of a multimodal emotion recognition brain-computer interface instrument based on a contrastive learning gated network.

FIG. 2 is a diagram of a unimodal feature encoder pre-training module.

FIG. 3 is a diagram of a multimodal contrastive learning training module.

FIG. 4 is a diagram of a gated structure training module.

FIG. 5 shows experimental results of the present disclosure on a TJU (Tianjin University)-Emotion dataset.

FIG. 6 shows experimental results of the present disclosure on a public dataset Semantic Emotional EEG Dataset (SEED).

FIG. 7 is a diagram showing performance results of the present disclosure in an ablation experiment.

FIG. 8 is a confusion diagram of an EEG mode and an eye mode of the present disclosure.

FIG. 9 is a diagram showing experimental results of unimodal EEG and eye and two multimodal fusion methods of the present disclosure.

FIG. 10 shows two-dimensional projection results of four decomposed features of a test sample of the present disclosure.

### DETAILED DESCRIPTION OF THE EMBODIMENTS

In the following description, specific details such as particular system structures and technologies are provided for illustration purposes rather than limitation to thoroughly understand the embodiments of the present disclosure. However, those skilled in the art should be aware that the present disclosure can also be achieved in other embodiments without these specific details. In other cases, detailed descriptions of well-known systems, apparatuses, circuits, and methods are omitted to prevent unnecessary details from hindering the description of the present disclosure.

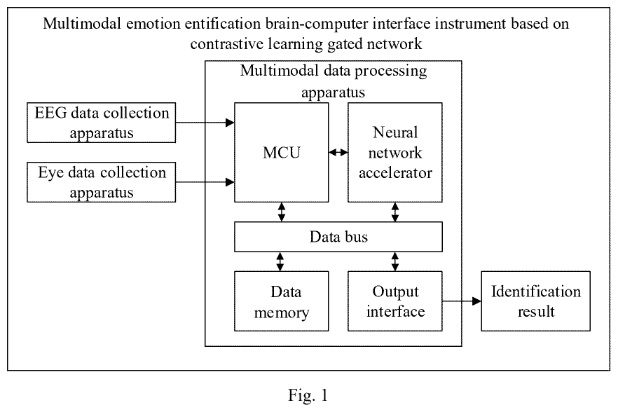

As shown in FIG. 1, the instrument is composed of an EEG data collection apparatus, an Eye data collection apparatus, and a multimodal data processing apparatus, wherein the multimodal data processing apparatus includes an MCU, a neural network accelerator, a data bus, a data memory, and an output interface. The EEG data collection apparatus is configured to collect EEG data, the eye movement data collection apparatus is configured to collect eye movement data, and the collected EEG data and eye movement data are stored into the data memory through the control of the MCU. Further, the MCU controls the neural network accelerator to acquire the EEG data and eye movement data from the data memory to train the multimodal contrastive learning gated network.

Further, the EEG data collection apparatus is composed of a power source, a main control chip, and an analog-to-digital converter.

Further, the eye movement data collection apparatus is composed of binocular infrared cameras, and the infrared cameras can capture clear eye images in low-light or no-light environments to avoid visible light interference.

In order to verify the practical effect of a multimodal collection helmet in emotion recognition, a multimodal emotion recognition method MCGNet combining EEG signals and eye movement signals is performed in the multimodal emotion recognition brain-computer interface instrument provided in the present disclosure. Specifically, the MCU controls the neural network accelerator to acquire the EEG data and eye movement data from the data memory to train the multimodal contrastive learning gated network. The training includes the following three modules: a unimodal feature encoder pre-training module, a multimodal contrastive learning training module, and a gated structure training module.

The unimodal feature encoder pre-training module is configured to respectively input EEG primary features and eye movement primary features into an EEG feature encoder and an eye movement feature encoder, and respectively input encoding results into a classifier to respectively predict unimodal emotion categories.

The multimodal contrastive learning training module is configured to respectively input EEG primary features and eye movement primary features into feature encoders of the unimodal feature encoder pre-training module to obtain multimodal features EEG and Eye, then respectively use an EEG similarity mapper and an EEG dissimilarity mapper as well as an eye similarity mapper and an eye dissimilarity mapper to decompose EEG and Eye signals, fuse decomposition results, and perform intra-sample contrastive learning and inter-sample contrastive learning.

The gated structure training module is configured to fuse EEG primary features and eye movement primary features and then input the fused EEG primary features and eye movement primary features into a gated network, the gated network generating a two-dimensional vector as an output to select and activate different expert network branches to complete emotion classification tasks.

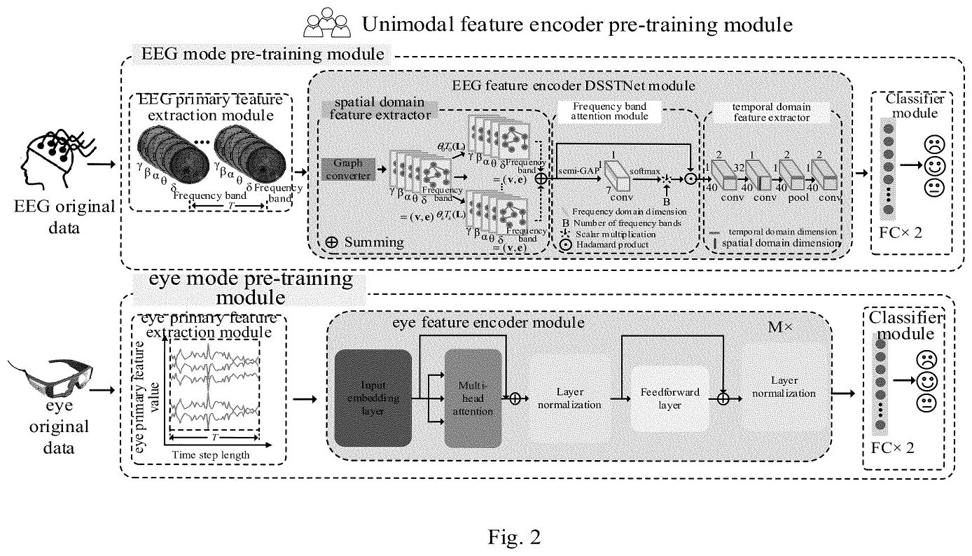

The unimodal feature encoder pre-training module constructed by the present disclosure is shown in FIG. 2. The pre-training module includes an EEG mode pre-training module and an eye movement mode pre-training module. The EEG mode pre-training module is composed of an EEG primary feature extraction module, an EEG feature encoder DSSTNet module, and a classifier module connected in sequence. The eye movement mode pre-training module is composed of an eye movement primary feature extraction module, an eye movement feature encoder module, and a classifier module connected in sequence.

Specifically, in the EEG mode pre-training module, at a pre-training stage, a sample is given, the EEG primary feature extraction module is configured to extract an EEG feature of sample data and input the feature into the EEG feature encoder DSSTNet module, and the output of the EEG feature encoder DSSTNet module is input into the classifier module to predict a category of a unimodal emotion. The EEG feature encoder DSSTNet module includes a spatial domain feature extractor, a frequency band attention module, and a temporal domain feature extractor connected in sequence. The spatial domain feature extractor is configured to extract spatial domain features of EEG signals, the frequency band attention module is configured to extract frequency domain features, and the temporal domain feature extractor is configured to extract temporal domain features.

Specifically, original EEG signals are preprocessed and then input into the spatial domain feature extractor, and the spatial domain feature extractor is composed of a graph converter and an adaptive graph convolution layer. In the graph converter, convolution kernels with a size of 64×1 and a step length of 1×1 are first used for convolution along an electrode channel dimension, thereby obtaining spatial domain features of the EEG signals. The purpose of setting a larger convolution kernel size is to obtain a wider receptive field, thereby fully extracting the spatial domain features. This layer uses a filling operation to maintain input and output feature dimensions unchanged. Subsequently, the obtained spatial domain features are represented as graph structure data.

Due to the irregular and discrete distribution of EEG channels, the adaptive graph convolution layer is used to extract space features of EEG signal channel dimensions. In a graph theory, a graph can be defined as G=(v,e) wherein v and e respectively represent a node and an edge in the graph. The connection relationship among N different nodes in the graph can be represented by an adjacency matrix A∈ [symbol] . Assuming a filter function g_{θ}=diag(θ), a graph convolution operation for a given spatial domain signal x∈R^{N} can be defined as:

$${g}_{θ} {*}_{G} x = Udiag ( θ ) {U}^{T} x , \qquad ( 1 )$$

wherein 0 is a variable parameter, *_{G} represents a graph convolution operation, and U∈ [symbol] is a standard orthogonal matrix composed of feature vectors of a Laplacian matrix L E [symbol] in a graph G. In an actual calculation process, it is difficult to directly calculate g_{θ}, so a K -order Chebyshev polynomial is used to approximately replace g_{θ}. The adaptive graph convolution layer can automatically update the adjacency matrix in a back propagation process without the need for manual construction. First, the adjacency matrix is initialized randomly, and when a back propagation operation is applied to adaptively learn an optimal adjacency matrix A*, a partial derivative of a loss function with respect to the A* is calculated, and the A* is updated according to the following rules:

$${A}^{*} = ( 1 - ρ ) {A}^{*} - ρ \frac{\partial Loss}{{A}^{*}} , \qquad ( 2 )$$

wherein ρ is a parameter for controlling the update rate of the adjacency matrix, and ρ=0.001 is set in the present disclosure.

The spatial domain feature extractor solves the problem of difficulty in effectively extracting spatial domain features of EEG signals, avoids interfering with the adjacency matrix, and can adaptively update the adjacency matrix for specific subjects.

Frequency band attention module: The module is located between the spatial domain feature extractor and the temporal domain feature extractor, and includes three parts: semi-global pooling, local cross-frequency band interaction, and adaptive weighting. First, semi-global pooling is used for global average pooling in a space dimension while retaining time dimension information. Subsequently, a convolution kernel of 7×1 is used to extract frequency domain features, and a filling operation is used to retain the temporal domain features and electrode channel dimensions of feature graphs. Compared to a fully connected layer, the convolution layer reduces the number of parameters and promotes the local interaction. Finally, the selected features are adaptively weighted, the frequency domain information is probabilistically transformed with a softmax function, and then, softmax output and input features are subjected to a Hadamard product.

Temporal domain feature extractor: A temporal domain feature extraction module is constructed by stacking a plurality of one-dimensional convolution layers. The first layer is composed of D convolution kernels with a size of 1×2 and a step length of 1×1 for convolution along a temporal domain dimension. The second layer of convolution is composed of D kernels with a size of 32×1 and a step length of 1×1, aiming to reduce the spatial domain dimension of features to 1 for better extraction of temporal domain information. Subsequently, a batch normalization method is used to alleviate the problem of gradient vanishing and accelerate a model training process. Then, an ELU activation function is used to nonlinearize the data. The third layer performs average pooling along a temporal domain dimension, and a convolution kernel has a size of 1×1 and a step length of 1×2. The pooling layer achieves the effect of preventing overfitting and reducing computational complexity while smoothing time features. The last layer further extracts temporal domain features by D kernels with a size of 1×2 and a step length of 1×1. The hyperparameter D in the present disclosure is set to 40.

Finally, the EEG feature graph is allowed to pass through a classifier module composed of two fully connected layers, and the output thereof is calculated with a softmax function to obtain prediction probabilities for each of three emotions.

Specifically, in the eye movement mode pre-training module, at a pre-training stage, a sample is given, the eye movement primary feature extraction module is configured to extract an eye feature of sample data and input the feature into the eye movement feature encoder module, and the output of the eye movement feature encoder module is input into the classifier module to predict a category of a unimodal emotion.

After original eye signals are preprocessed, differential entropy features of primary features are respectively extracted in frequency bands of [0, 0.2] Hz, [0.2, 0.4] Hz, [0.4, 0.6] Hz, and [0.6, 1] Hz. The features extracted from the left and right eyes include a mean value, a standard deviation, and differential entropy features of four frequency bands, which are used as inputs of the eye movement feature encoder module, represented as FT×K_{Eye}, wherein the length of a sliding window is T, and the feature dimension is K.

The eye movement feature encoder module uses the structural design of an encoder in a transformer architecture, including an input embedding layer, multi-head attention, layer normalization, and a feedforward layer. The advantage of the transformer is to simultaneously process long-distance dependency relationship and capture global contextual information, and effectively learn the internal structure and key features within an input sequence through a self-attention mechanism.

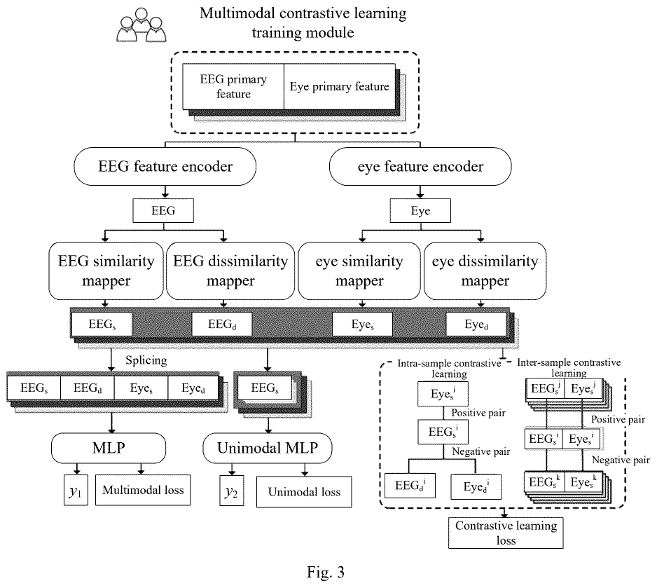

The multimodal contrastive learning training module constructed by the present disclosure is shown in FIG. 3. In this module, EEG primary features and eye movement primary features are respectively input into encoders of the unimodal feature encoder pre-training module to obtain multimodal features EEG and Eye; subsequently, an EEG similarity mapper and an EEG dissimilarity mapper are configured to decompose the multimodal feature EEG into a similar feature EEGₛ and a dissimilar feature EEG_{d}, and an eye similarity mapper and an eye dissimilarity mapper are configured to decompose the multimodal feature Eye into a similar feature Eyeₛ and a dissimilar feature Eye_{d}; and then, the four decomposed features are fused in different splicing manners, and intra-sample contrastive learning and inter-sample contrastive learning are performed. The present disclosure provides two splicing manners. First manner: Splicing is performed according to a feature dimension, and splicing results are output into an MLP classifier to obtain an emotion prediction label (i.e., y₁ in FIG. 3). Second manner: Splicing is performed according to a batch dimension, and splicing results are respectively input into a weight-shared MLP classifier to obtain four unimodal prediction labels represented by y₂.

In the first manner, a dataset is denoted as M, for a given sample i∈M, multimodal emotion classification loss [symbol] is calculated by cross entropy (CE) loss, and calculation methods are as follows:

$${y}_{1}^{i} = MLP ( [ EE {G}_{s} ; {Eye}_{s} ; {EEG}_{d} ; {Eye}_{d} ] ) , \qquad ( 3 )$$

$$\text{?} = CE ( {y}^{i} , {y}_{1}^{i} ) , \qquad ( 4 )$$

wherein [;] represents splicing according to a feature dimension, and yⁱ represents a true label of an emotion category.

In the second manner, unimodal emotion classification loss [symbol] is:

$${y}_{2}^{i} = MLP ( [ {EEG}_{s} ; {Eye}_{s} ; {EEG}_{d} ; {Eye}_{d} ] ) , \qquad ( 5 )$$

$$\text{?} {i}_{{2}_{uni}}^{1} , \qquad ( 6 )$$

wherein [,] represents splicing according to a batch dimension, yⁱ represents a true label of unimodal prediction, and the four decomposed features are separately predicted.

In the intra-sample contrastive learning and inter-sample contrastive learning, positive and negative sample pairs are constructed by the four decomposed features, as shown in FIG. 3. First, for a sample pair (i,j) given by the dataset M, features are extracted by feature encoders, and a cosine similarity score between a sample [EEGⁱ; Eyeⁱ] and a sample [EEG^{j}; Eye^{j}] is calculated:

$${𝒞}^{i , j} = sim ( [ {EEG}^{i} ; {Eye}^{i} ] , [ {EEG}^{j} ; {Eye}^{j} ] ) . \qquad ( 7 )$$

Then, similar samples and dissimilar samples are selected for a sample i. The samples with labels the same as the label yⁱ are sorted in a descending order of cosine similarity scores calculated according to the previous step, so as to construct a candidate similar sample set

$${S}_{0}^{i} .$$

Furthermore, the samples with labels different from the label yⁱ are divided into a candidate dissimilar sample set

$${S}_{1}^{i} .$$

Two samples with relatively high cosine similarity scores are randomly selected from the set

$${S}_{0}^{i}$$

and are combined with the sample i to form an inter-sample positive pair, denoted as Neighborⁱ. Two samples with relatively low cosine similarity scores are randomly selected from the set

$${S}_{1}^{i} ,$$

denoted as

$${Outlier}_{1}^{i} .$$

Two samples with relatively high cosine similarity scores are randomly selected, denoted as

$${Outlier}_{2}^{i} . {Outlier}_{1}^{i} and {Outlier}_{2}^{i}$$

are combined with the sample i to form an inter-sample negative pair Outlier.

First, an intra-sample positive pair

$${𝒫}_{i n t r a}^{i}$$

and an intra-sample negative pair

$${𝒩}_{intra}^{i}$$

are constructed:

$${𝒫}_{intra}^{i} = ( {EEG}_{s}^{i} , {Eye}_{s}^{i} ) ⋃ ( {EEG}_{s}^{j} , {Eye}_{s}^{j} ) , \qquad ( 8 )$$

$${𝒩}_{intra}^{i} = \{ ( {EEG}_{s}^{i} , {EEG}_{d}^{i} ) , ( {EEG}_{s}^{i} , {Eye}_{d}^{i} ) \} ⋃ \{ ( {EEG}_{s}^{j} , {EEG}_{d}^{j} ) , ( {EEG}_{s}^{j} , {Eye}_{d}^{j} ) \} \qquad ( 9 )$$

wherein j∈Neighborⁱ∪Outlierⁱ, Neighborⁱ, and Outlierⁱ respectively represent similar and dissimilar samples of the sample i.

Then, an inter-sample positive pair

$${𝒫}_{i n t e r}^{i}$$

and an inter-sample negative pair

$${𝒩}_{inter}^{i}$$

are constructed as follows:

$${𝒫}_{inter}^{i} = ( {EEG}_{s}^{i} , {Eye}_{s}^{j} ) ⋃ ( {Eye}_{s}^{j} , {Eye}_{s}^{j} ) , \qquad ( 10 )$$

$${𝒩}_{inter}^{i} = ( {EEG}_{s}^{i} , {EEG}_{s}^{k} ) ⋃ ( {Eye}_{s}^{j} , {Eye}_{s}^{k} ) , \qquad ( 11 )$$

$$wherein j ∈ {Neighbor}^{i} , and k ∈ {Outlier}^{i} .$$

Loss functions of intra-sample contrastive learning and inter-sample contrastive learning are represented by joint contrastive loss, including two aspects: comparison between inter-sample similar samples and dissimilar samples, and comparison between intra-sample similar features and dissimilar features. For a given sample i, the contrastive learning loss Lₑ is:

$$\text{?} = \frac{1}{n} {\sum }_{i = 1}^{n} \text{?} . \qquad ( 12 )$$

In an NT-Xent contrastive learning loss framework, intra-sample contrastive learning is combined with inter-sample contrastive learning to perform modal feature decomposition and modal representation learning. The loss of the sample i is represented as:

$$\text{?} = {\sum }_{( a , p ) ∈ {𝒫}^{i}} - log \frac{exp ( si m ( a , p ) / τ}{{\sum }_{( a , n ) ∈ {𝒩}^{i} ⋃ {𝒫}^{i}} exp ( si m ( a , p ) / τ )} , \qquad ( 13 )$$

wherein (a, p) and (a, n) represent a pair of intra-sample decomposed feature vectors, such as

$$( EE {G}_{s}^{{iEye}_{s}^{i}} and ( {EEG}_{d}^{{iEye}_{d}^{i}} ,$$

or a pair of inter-sample decomposed feature vectors, such as

$$( {EEG}_{s}^{{iEEG}_{s}^{j}} .$$

Pⁱ is a positive pair set, represented as

$${P}^{i} = {P}_{intra}^{i} ⋃ {P}_{inter}^{i} ,$$

including an intra-sample positive pair

$${P}_{intra}^{i}$$

and an inter-sample positive pair

$${P}_{intra}^{i} .$$

Nᵢ is a negative pair set, represented as

$${N}^{i} = {N}_{intra}^{i} ⋃ {N}_{inter}^{i} ,$$

including an intra-sample negative pair

$${N}_{intra}^{i}$$

and an inter-sample negative pair

$${N}_{inter}^{i} .$$

(a, p) is a positive pair in Pⁱ, and (a, n) is a negative pair in Nⁱ.

The total loss function of this module is:

[symbol]

$$ℒ = {ℒ}_{p r e d} + {λ}_{uni} {ℒ}_{uni} + {λ}_{c} {L}_{c} , \qquad ( 14 )$$

wherein [symbol] represents multimodal emotion classification loss, [symbol] represents unimodal emotion classification loss, and [symbol] represents contrastive learning loss. λᵤₙᵢ and λ_{c} determine the degree of contribution of each task to the update of model parameters during a training process.

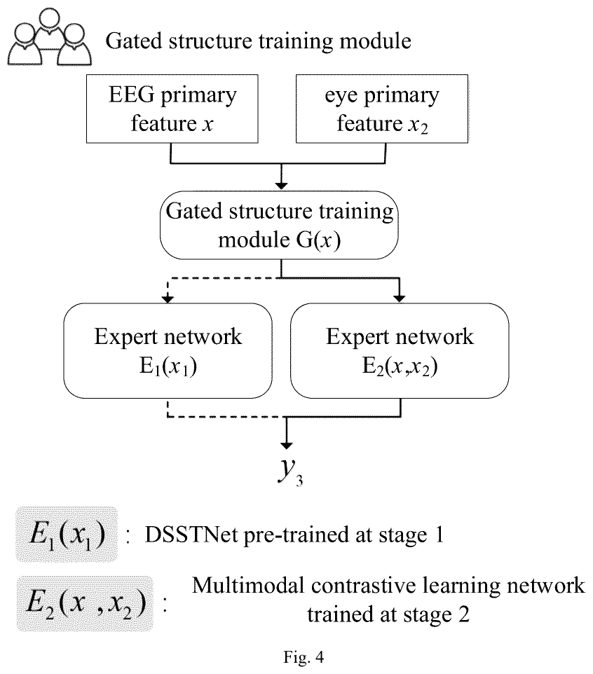

The gated structure training module constructed by the present disclosure is shown in FIG. 4. In this module, an EEG primary feature x₁ and an eye primary feature x₂ are spliced and then input into a gated network G(x) formed by an MLP. The gated network generates a two-dimensional vector g as an output to select and activate different network branches to complete emotion classification tasks. A group of expert networks are selected according to a hybrid expert model. Each expert specializes in studying subsets of all modes. In specific tasks, two modes at most can have three expert networks, represented as E₁(x₁), E₂(x₁,x₂), and E₃(x₂) respectively. The combination of EEG and eye can provide useful clues, but in practical uses, sometimes unimodal EEG has a better ability to identify emotions. In the present disclosure, a unimodal EEG emotion classification network model pre-trained by the unimodal feature encoder pre-training module serves as an expert network 1 E₁(x₁), and a multimodal contrastive learning network model trained by the multimodal contrastive learning training module serves as an expert network 2 E₂(x₁, x₂). A finally output prediction label is represented by y₃.

$${y}_{3} = {g}_{1} {E}_{1} ( {x}_{1} ) + {g}_{2} {E}_{2} ( {x}_{1} , {x}_{2} ) . \qquad ( 15 )$$

The complexities of the two expert network models selected in the present disclosure are different. Usually, the expert network with a high model complexity has a relatively strong representation ability. If the network is trained only by minimizing the loss of emotion classification tasks, the gated network will always select a branch with a high model complexity, so an additional term loss function β[g₁C(E₁)+g₂C(E₂)] is also introduced when a loss function is designed, wherein g₁ and g₂ represent decision vectors output by a gated structure, C(E) represents the cost of executing an expert network (such as MAdds), and β is a hyperparameter for adjusting the relative importance between two loss terms.

The present disclosure jointly optimizes the expert network and the gated network in an end-to-end manner. For a logic vector output by the gated structure MLP, a softmax function with a temperature parameter is used for processing to adjust the sensitivity of a decision boundary. In addition, the present disclosure relates to selecting different expert networks for subsequent training, so that each network focuses on a specific mode of data.

The present disclosure uses a self-built dataset (TJU-Emotion) and a publicly available SEED dataset to evaluate the performance of the provided method. Experimental results show that the performance of the present disclosure in emotion recognition tasks is superior to a series of most advanced contrastive methods.

First, the present disclosure constructs a three-classified multimodal emotion dataset, named a TJU-Emotion dataset, including EEG and eye multimodal data. Experiments are conducted at Tianjin University, and a total of 10 subjects are invited to participate in experiments. The EEG mode selects 32 electrodes for an experiment, and the eye data uses the pupil diameter data of left and right eyes of the subjects. The experiment uses 15 movie clips (which can cause positive, neutral and negative emotions) as stimuli, and the experiment includes 15 trials.

The pre-processing steps of the EEG part include filtering, reduction of a sampling rate, re-referencing conversion, segmentation, and artifact removal. The pre-processed EEG data will be divided into samples with a sliding window length T=15s and a step length ΔT=1s. The pre-processing of the eye part utilizes principal component analysis pairs to remove first principal component light reflection.

The publicly available SEED dataset includes EEG data of 15 subjects watching 15 Chinese movie clips with negative, positive and neutral emotions. The EEG electrode distribution uses an international 10-20 electrode positioning solution with a total of 62 electrode channels.

A total of four quantitative and qualitative effect analyses are conducted on the present disclosure: contrastive experiments with most advanced methods, influence of each structure on model performance, multimodal information complementarity analysis, and contrastive learning visualized analysis.

The present disclosure implements subject dependent experiments, and calculates the average classification accuracy, standard deviation and F1-score of all subjects. During an experiment, the learning rate of an Adam optimizer is set to 1e-5 and 1e-3 respectively in two datasets, and parameters λᵤₙᵢ, λ_{c} and β are set to 0.1.

The present disclosure selects six baseline models to compare the performance of the present disclosure, including a maximum rule MAX and a sum rule SUM of a simple decision-level fusion method, a model fusion method Fuzzy based on fuzzy integrals, a deep canonical correlation analysis method DCCA-AM with an attention mechanism, a method BAT that uses a bidirectional adapter to transmit complementary information among different modes, and a contrastive learning and feature reconstruction method CARAT.

First, a unimodal feature encoder is configured to perform a unimodal emotion classification experiment on 10 subjects in the TJU-Emotion dataset, and the performance of the present disclosure is compared using the six baseline models mentioned in the previous section. Experimental results are shown in FIG. 5.

From the first two columns of the figure, it can be seen that the unimodal emotion classification performance depends on the used data type, and the EEG mode is the best in the single mode. The model of the present disclosure significantly improves the performance of emotion classification by combining multimodal information. Subsequently, six contrastive experiments of a multimodal fusion method are conducted in the dataset, and t-test significance p-values of several models relative to the present disclosure are calculated. The classification performance of the present disclosure shows significant differences compared to other methods. Experimental results show that the present disclosure not only has significant advantages in classification accuracy, but also exhibits significant advantages in model stability. In addition, the average F1-score of the present disclosure is improved compared to other methods.

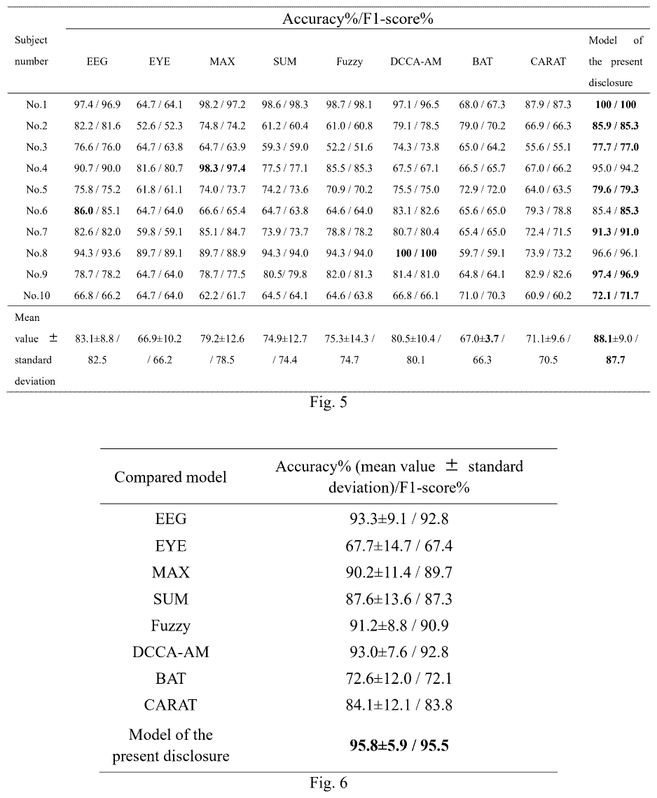

The performance of the provided method is also verified on the widely used public dataset SEED. Experimental results are shown in FIG. 6, indicating that the provided method is also competitive on the public dataset.

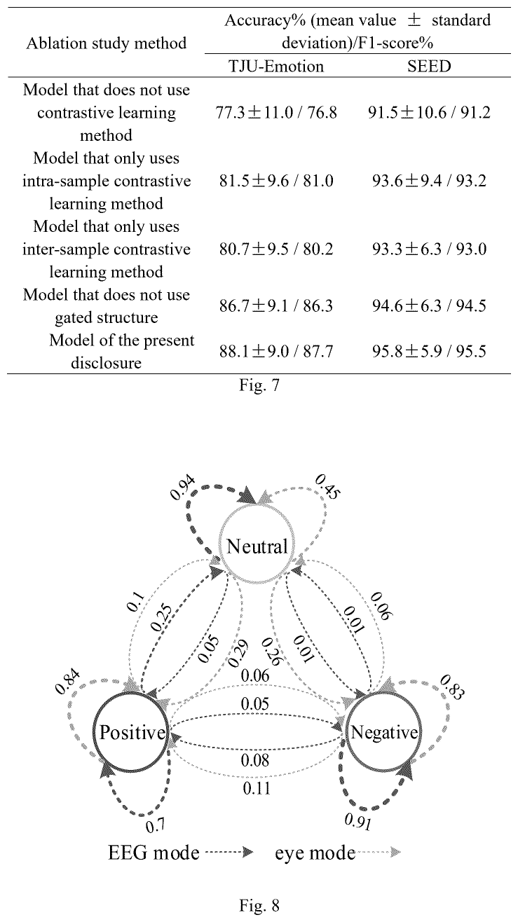

In order to investigate the influence of each structure on the performance of the present disclosure, several ablation experiments are set, and results are shown in FIG. 7. Models of the following ablation experiments are included: a model that does not use a contrastive learning method, a model that only uses an intra-sample contrastive learning method, a model that only uses an inter-sample contrastive learning method, and a model that does not use a gated structure.

For the TJU-Emotion dataset, it can be seen from FIG. 7 that the performance of the models of the four ablation experiments is inferior to that of the model of the present disclosure. Compared to the model that does not use a contrastive learning method, the performance of the model that uses an intra-sample contrastive learning or inter-sample contrastive learning method is significantly improved. Moreover, the model of the present disclosure has a more flexible structure, and different modes and expert networks can be dynamically selected according to the features and needs of each sample. Similar conclusions are also obtained for the SEED dataset.

FIG. 8 shows a confusion diagram of an EEG mode and an eye mode. In the eye mode, the identification accuracy of a positive state is higher than that in the EEG mode. The EEG mode is better at identifying neutral and negative states. Therefore, it is expected that the two modes can complement each other to improve the performance of identifying each emotion state.

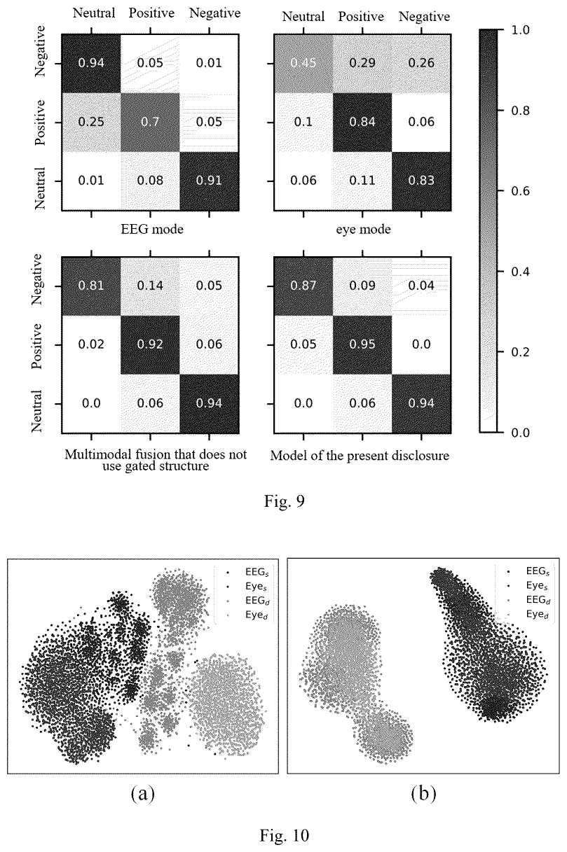

FIG. 9 shows unimodal EEG and eye and two multimodal fusion methods: a confusion matrix of a model that does not use a gated structure and a model of the present disclosure, showing the advantages and disadvantages of different modes and different mode fusion methods. It is found that the EEG and eye modes have important complementary features, and the multimodal fusion method significantly improves the classification performance. On the whole, the effective combination of multiple modes can utilize the characteristics and complementarity of each mode to improve the ability to identify three types of emotions.

In the TJU-Emotion, a subject is randomly selected, and two-dimensional projections of four decomposed features of all test samples are shown in FIG. 10, wherein FIG. 10(a) does not use a contrastive learning method, and FIG. 10(b) uses a contrastive learning method. It can be seen from the figure that after the contrastive learning method is used, similar features (EEGₛ and Eyeₛ) become closer and closer, while dissimilar features (EEG_{d} and Eye_{d}) become farther and farther away from the corresponding similar features thereof. This indicates the effectiveness of using the contrastive learning method to learn consistency and inconsistency between modes.

It should be understood that the size of the sequence number of each step in the above embodiments does not imply the order of execution, and the order of execution of each process should be determined according to the functions and internal logics thereof, and should not constitute any limitation on the implementation processes of the embodiments in the present disclosure.

Those skilled in the art can clearly understand that for the sake of convenience and simplicity in description, only the division of the functional units and modules described above will be illustrated with examples. In practical uses, the above function allocation can be completed by different functional units or modules according to needs, that is, the internal structure of the apparatus is divided into different functional units or modules to complete all or part of the functions described above. The functional units or modules in the embodiments can be integrated into one processing unit, or each of the units can exist alone physically, or two or more units can be integrated into one unit. The above integrated unit can be implemented in the form of hardware, or can be implemented in the form of a software functional unit. In addition, the specific name of each functional unit or module is only for the convenience of distinguishing from each other, and is not intended to limit the scope of protection of the present disclosure. The specific working processes of the units or modules in the above system can refer to the corresponding processes in the above method embodiment, and will not be repeated here.

In the above embodiments, the description of each embodiment has an emphasis, and the parts that are not described in detail or recorded in a certain embodiment can refer to the relevant descriptions of other embodiments.

Those of ordinary skill in the art can realize that the units and algorithm steps of each example described with reference to the embodiments disclosed herein can be implemented in the form of electronic hardware, or a combination of computer software and electronic hardware. Whether the functions are executed in a mode of hardware or software depends on particular uses and design constraint conditions of the technical solutions. Those skilled in the art can implement the described functions by using different methods for each specific use, but such implementation should not be considered beyond the scope of the present disclosure.

In the embodiments provided by the present disclosure, it should be understood that the disclosed apparatus/terminal device and method can be implemented in other modes. The embodiments of the apparatus/terminal device described above are only illustrative. The division of the modules or units is only a logical function division. In an actual implementation, there can be other division methods. A plurality of units or components can be combined or can be integrated into another system, or some features can be ignored or not implemented. In addition, the displayed or discussed mutual couplings or direct couplings or communication connections can be implemented through some interfaces, and the indirect couplings or communication connections between apparatuses or units can be implemented in electrical, mechanical, or other forms.

The units described as separate components can or cannot be physically separated, and components displayed as units can or cannot be physical units, i.e., can be located in one place, or can be distributed on a plurality of network units. Some or all of the units can be selected according to actual needs to achieve the objectives of the solution of this embodiment.

In addition, each functional unit in each embodiment of the present disclosure can be integrated into one processing unit, each unit can separately exist physically, or two or more units can be integrated into one unit. The above integrated unit can be implemented in the form of hardware, or can be implemented in the form of a software functional unit.

When the integrated module/unit is implemented in the form of a software functional unit and sold or used as an independent product, the integrated module/unit can be stored in a computer-readable storage medium. Based on this understanding, all or part of the processes in the above embodiment methods implemented by the present disclosure can also be completed by instructing related hardware through a computer program. The computer program can be stored in a computer-readable storage medium. The computer program, when executed, by a processor can implement the steps in each of the above method embodiments. The computer program includes computer program code, and the computer program code can be in the form of source code, target code or an executable file, or in some intermediate forms. The computer-readable medium can include: any entity or apparatus capable of carrying the computer program code, a recording medium, a USB flash drive, a portable hard drive, a magnetic disk, an optical disk, a computer memory, a read-only memory (ROM), a random access memory (RAM), an electric carrier signal, a telecommunication signal, a software distribution medium, etc. It should be noted that the content included in the computer-readable medium can be appropriately added or deleted according to requirements of the legislation and patent practice in a jurisdiction. In some jurisdictions, according to the legislation and patent practice, the computer-readable medium does not include an electric carrier signal and a telecommunication signal.

The above embodiments are only used for describing the technical solutions of the present disclosure, but are not intended to limit the present disclosure. Although the present disclosure is described in detail with reference to the above embodiments, those of ordinary skill in the art should understand that modifications can still be made to the technical solutions described in the above embodiments or equivalent replacements can be made to some technical features, and these modifications or replacements do not make the essence of the corresponding technical solutions depart from the spirit and scope of the technical solutions of the embodiments of the present disclosure, and should be included within the scope of protection of the present disclosure.

What is claimed is:

1. A multimodal emotion recognition instrument based on a contrastive learning gated network, wherein the instrument comprises an electroencephalogram (EEG) data collection apparatus configured to collect EEG data, an eye movement data collection apparatus configured to collect eye movement data, and a multimodal data processing apparatus;
the multimodal data processing apparatus comprises a microprogrammed control unit (MCU), a neural network accelerator, a data bus, a data memory, and an output interface; and the MCU controls the neural network accelerator to acquire EEG data and eye movement data from the data memory to train the multimodal contrastive learning gated network, specifically comprising:
a unimodal feature encoder pre-training module, configured to respectively input EEG primary features and eye movement primary features into an EEG feature encoder and an eye movement feature encoder, and respectively input encoding results into a classifier to respectively predict unimodal emotion categories;
a multimodal contrastive learning training module, configured to respectively input EEG primary features and eye movement primary features into feature encoders of the unimodal feature encoder pre-training module to obtain multimodal features EEG and Eye, then respectively use an EEG similarity mapper and an EEG dissimilarity mapper as well as an eye similarity mapper and an eye dissimilarity mapper to decompose EEG and eye movement signals, fuse decomposition results, and perform intra-sample contrastive learning and inter-sample contrastive learning; and
a gated structure training module, configured to fuse EEG primary features and eye movement primary features and then input the fused EEG primary features and eye movement primary features into a gated network, the gated network generating a two-dimensional vector as an output to select and activate different expert network branches to complete emotion classification tasks.

2. The multimodal emotion recognition instrument based on the contrastive learning gated network according to claim 1, wherein the unimodal feature encoder pre-training module comprises an EEG mode pre-training module and an eye movement mode pre-training module; the EEG mode pre-training module comprises an EEG primary feature extraction module, an EEG feature encoder DSSTNet module, and a classifier module connected in sequence; the eye movement mode pre-training module comprises an eye movement primary feature extraction module, an eye movement feature encoder module, and a classifier module connected in sequence; and
the EEG feature encoder DSSTNet module comprises a spatial domain feature extractor, a frequency band attention module, and a temporal domain feature extractor connected in sequence.

3. The multimodal emotion recognition instrument based on the contrastive learning gated network according to claim 1, wherein the multimodal contrastive learning training module is specifically configured to:
use the EEG similarity mapper and the EEG dissimilarity mapper to decompose a multimodal feature EEG into a similar feature EEGₛ and a dissimilar feature EEG_{d};
use the eye similarity mapper and the eye dissimilarity mapper to decompose a multimodal feature Eye into a similar feature Eyeₛ and a dissimilar feature Eye_{d}; and
fuse the four decomposed features at a feature dimension or a batch dimension, and perform intra-sample contrastive learning and inter-sample contrastive learning.

4. The multimodal emotion recognition instrument based on the contrastive learning gated network according to claim 1, wherein in the gated structure training module, a unimodal EEG emotion classification network model pre-trained by the unimodal feature encoder pre-training module serves as an expert network 1 E₁(x₁), a multimodal contrastive learning network model trained by the multimodal contrastive learning training module serves as an expert network 2 E₂(x₁, x₂), and a finally output prediction label is represented by y₃:
$${y}_{3} = {g}_{1} {E}_{1} ( {x}_{1} ) + {g}_{2} {E}_{2} ( {x}_{1} , {x}_{2} ) , \qquad ( 1 )$$
wherein g₁ and g₂ represent decision vectors output by a gated structure.

5. The multimodal emotion recognition instrument based on the contrastive learning gated network according to claim 2, wherein the spatial domain feature extractor is configured to extract spatial domain features of EEG signals, the frequency band attention module is configured to extract frequency domain features, and the temporal domain feature extractor is configured to extract temporal domain features.

6. The multimodal emotion recognition instrument based on the contrastive learning gated network according to claim 2, wherein the eye movement feature encoder module uses the structural design of an encoder in a transformer architecture, comprising an input embedding layer, multi-head attention, layer normalization, and a feedforward layer.

7. The multimodal emotion recognition instrument based on the contrastive learning gated network according to claim 3, wherein during fusion according to a feature dimension, a dataset is denoted as M, for a given sample i∈M, multimodal emotion classification loss L_{pred} is calculated by cross entropy loss, and calculation methods are as follows:
$${y}_{1}^{i} = MLP ( [ {EEG}_{s} ; {Eye}_{s} ; {EEG}_{d} ; {Eye}_{d} ] ) , \qquad ( 2 )$$
$${L}_{p r e d} = CE ( {y}^{i} , {y}_{1}^{i} ) , \qquad ( 3 )$$
wherein [;] represents splicing according to a feature dimension, yⁱ represents a true label of an emotion category, and MLP represents a multi-layer perceptron.

8. The multimodal emotion recognition instrument based on the contrastive learning gated network according to claim 3, wherein during fusion according to a batch dimension, unimodal emotion classification loss Lᵤₙᵢ is:
$${y}_{2}^{i} = MLP ( [ {EEG}_{s} ; {Eye}_{s} ; {EEG}_{d} ; {Eye}_{d} ] ) , \qquad ( 4 )$$
$${L}_{u n i} = CE ( {y}^{i} , {y}_{2}^{i} ) , \qquad ( 5 )$$
wherein [,] represents splicing according to a batch dimension, yⁱ represents a true label of unimodal prediction, and MLP represents a multi-layer perceptron.

9. The multimodal emotion recognition instrument based on the contrastive learning gated network according to claim 3, wherein in the intra-sample contrastive learning and inter-sample contrastive learning, positive and negative sample pairs are constructed by the four decomposed features, specifically comprising:
for a sample pair (i,j) given by the dataset M, features being extracted by feature encoders, and a cosine similarity score between a sample [EEGⁱ; Eyeⁱ] and a sample [EEG^{j}; Eye^{j}] being calculated:
$${𝒞}^{i , j} = sim ( [ {EEG}^{i} ; {Eye}^{i} ] , [ {EEG}^{j} ; {Eye}^{j} ] ) ; \qquad ( 6 )$$
similar samples and dissimilar samples being selected for a sample i; the samples with labels the same as the label yⁱ being sorted in a descending order of cosine similarity scores calculated according to Formula (6), so as to construct a candidate similar sample set
$${S}_{0}^{i} ;$$
furthermore, the samples with labels different from the label yⁱ being divided into a candidate dissimilar sample set
$${S}_{1}^{i} ;$$
two samples with relatively high cosine similarity scores being randomly selected from the set
$${S}_{0}^{i}$$
and being combined with the sample i to form an inter-sample positive pair, denoted as Neighborⁱ; two samples with relatively low cosine similarity scores being randomly selected from the set
$${S}_{1}^{i} ,$$
denoted as
$${Outlier}_{1}^{i} ;$$
two samples with relatively high cosine similarity scores being randomly selected, denoted as
$${Outlier}_{2}^{i} ; {Outlier}_{1}^{i} and {Outlier}_{2}^{i}$$
being combined with the sample i to form an inter-sample negative pair Outlierⁱ;
firstly, an intra-sample positive pair
$${𝒫}_{i n t r a}^{i}$$
and an intra-sample negative pair
$${𝒩}_{intra}^{i}$$
being constructed:
$${𝒫}_{intra}^{i} = ( {EEG}_{s}^{i} , {Eye}_{s}^{i} ) ⋃ ( {EEG}_{s}^{j} , {Eye}_{s}^{j} ) , \qquad ( 7 )$$
$${𝒩}_{intra}^{i} = \{ ( {EEG}_{s}^{i} , {EEG}_{d}^{i} ) , ( {EEG}_{s}^{i} , {Eye}_{d}^{i} ) \} ⋃ \{ ( {EEG}_{s}^{j} , {EEG}_{d}^{j} ) , ( {EEG}_{s}^{j} , {Eye}_{d}^{j} ) \} , \qquad ( 8 )$$
wherein j∈Neighborⁱ∪Outlierⁱ, Neighborⁱ, and Outlierⁱ respectively represent similar and dissimilar samples of the sample i; and
an inter-sample positive pair
$${𝒫}_{i n t e r}^{i}$$
and an inter-sample negative pair
$${𝒩}_{inter}^{i}$$
being constructed as follows:
$${𝒫}_{inter}^{i} = ( {EEG}_{s}^{i} , {Eye}_{s}^{j} ) ⋃ ( {Eye}_{s}^{j} , {Eye}_{s}^{j} ) , \qquad ( 9 )$$
$${𝒩}_{inter}^{i} = ( {EEG}_{s}^{i} , {EEG}_{s}^{k} ) ⋃ ( {Eye}_{s}^{j} , {Eye}_{s}^{k} ) , \qquad ( 10 )$$
$$wherein j ∈ {Neighbor}^{i} , and k ∈ {Outlier}^{i}$$

10. The multimodal emotion recognition instrument based on the contrastive learning gated network according to claim 1, wherein the EEG data collection apparatus comprises a power source, a main control chip, and an analog-to-digital converter; and the eye movement data collection apparatus comprises binocular infrared cameras.
